(19) United States
(12) Patent Application Publication
(10) Pub. No.: US 2017/0160058 A1
(43) Pub. Date: Jun. 8, 2017

(54) Load Distribution Garment

(71) Applicant: Safariland, LLC, Jacksonville, FL (US)

(72) Inventors: Orr Limpisvasti, Manhattan Beach, CA (US); David Pittman, Temecula, CA (US)

(21) Appl. No.: 15/239,247

(22) Filed: Aug. 17, 2016

Related U.S. Application Data
(60) Provisional application No. 62/206,914, filed on Aug. 19, 2015.

(51) Int. Cl. F41H 1/02; A41F 17/00; A41B 1/08; A41D 1/04

(52) U.S. Cl. CPC F41H 1/02; A41D 1/04; A41F 17/00; A41B 1/08; A41D 2400/80; A41B 2400/80; A61F 5/026

(57) ABSTRACT

A load distribution garment for use under a ballistic vest includes a shirt to be worn under the vest. The shirt has interface grips on its outside to engage the inside of the vest and help to transfer vertical load of the vest to the shirt. The shirt has tightenable straps that help to keep the shirt snub on the user's torso. As a result, some load of the vest is transferred to the user's torso, including the core, rather than being carried solely on the shoulders and collarbone area.

### BACKGROUND OF THE INVENTION

A ballistic vest can be relatively heavy for a law enforcement officer to wear, especially for an extended period of time such as an all-day shift. Also, the load (weight) of the vest is typically earned primarily by the shoulder straps of the vest, bearing down on the officer's shoulders. Together, these factors can create discomfort or pain, and can tire the wearer.

This invention relates to a load distribution garment. In particular, this invention relates to a load distribution garment, in the form of a shirt, that can be worn under a ballistic vest, to help distribute the load (weight) of the vest to the wearer's core. This invention also relates to a combination of a ballistic vest and a load distribution garment.

### BRIEF DESCRIPTION OF THE DRAWINGS

FIG. 1 is a front perspective view of a load distribution garment that is an embodiment of the invention;

FIG. 2 is a back perspective view of the load distribution garment of FIG. 1;

FIG. 3 is a view similar to FIG. 1 and showing also a ballistic vest worn over the load distribution garment;

FIG. 4 is a view similar to FIG. 1 and showing also a ballistic vest worn over the load distribution garment; and

FIG. 5 is a schematic illustration of the engagement of an interface grip on the load distribution garment with the inside of the vest.

### DETAILED DESCRIPTION

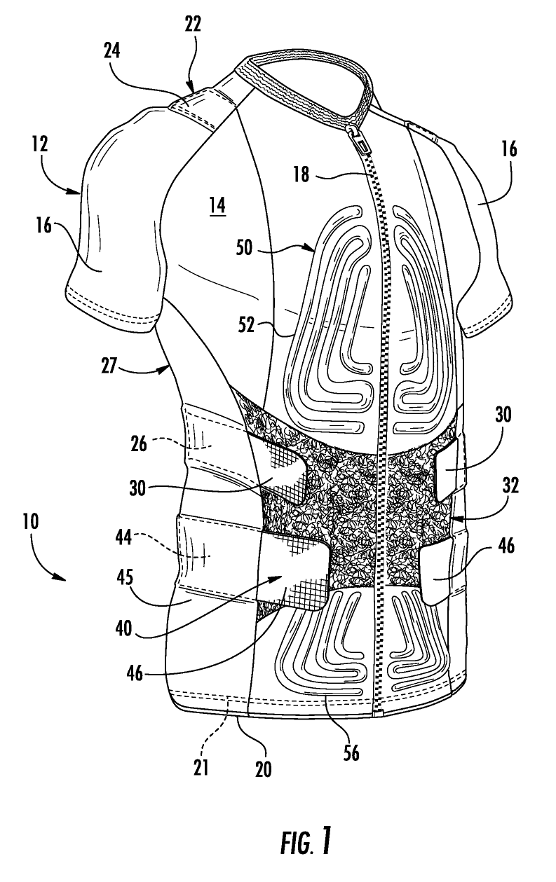

This invention relates to a load distribution garment that helps to remove some of the load of a ballistic vest from the wearer's shoulders and place that load, instead, on the wearer's core. The invention is applicable to many varied types of garments. As representative of the invention, FIG. 1 illustrates a load distribution garment 10 that is a first embodiment of the invention.

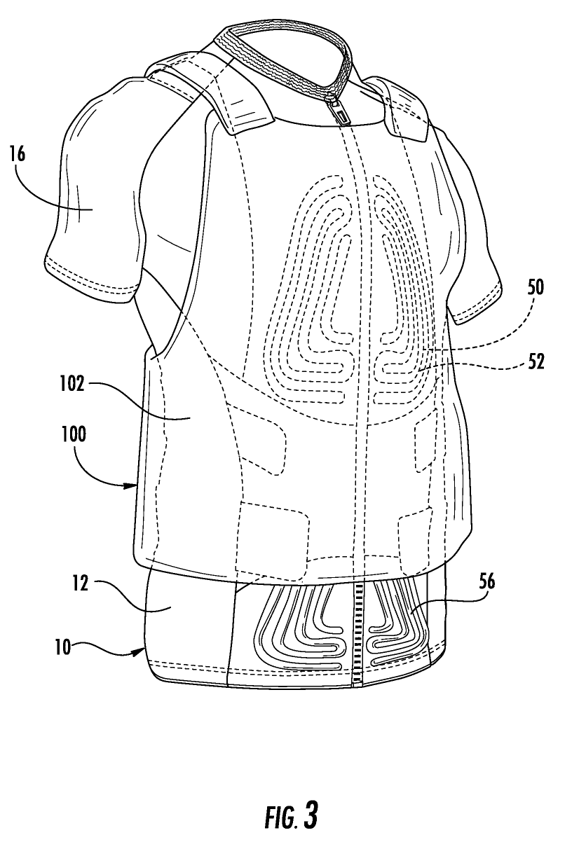

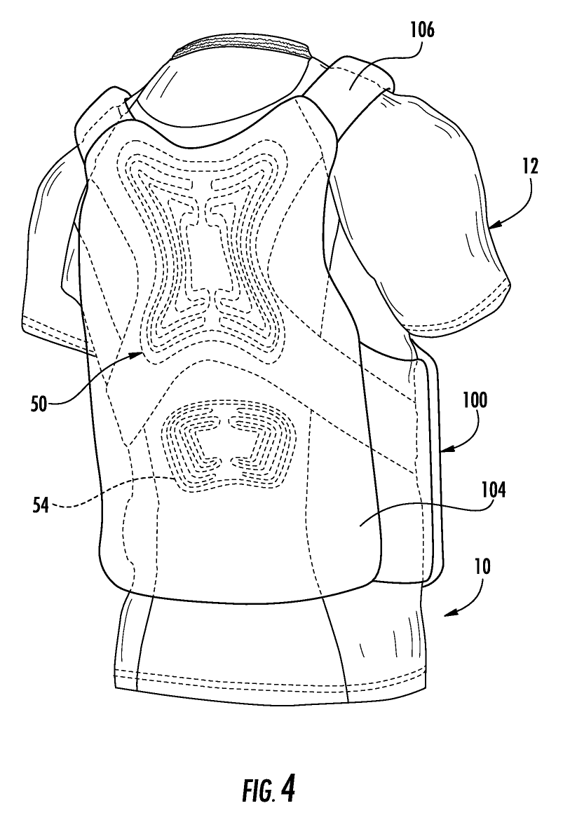

The garment 10 is typically used in combination with, and is configured for use with, a ballistic vest 100 (FIGS. 3 and 4). The ballistic vest 100 is shown only schematically in the drawings, since its construction for purposes of this invention is not novel. That is, the garment 10 is usable with most if not all known ballistic vests. The particular vest 100 that is illustrated includes a front panel 102 and a back panel 104 joined by shoulder straps 106. Each portion of the vest 100 has an inside surface that is presented toward the user when the vest is being worn.

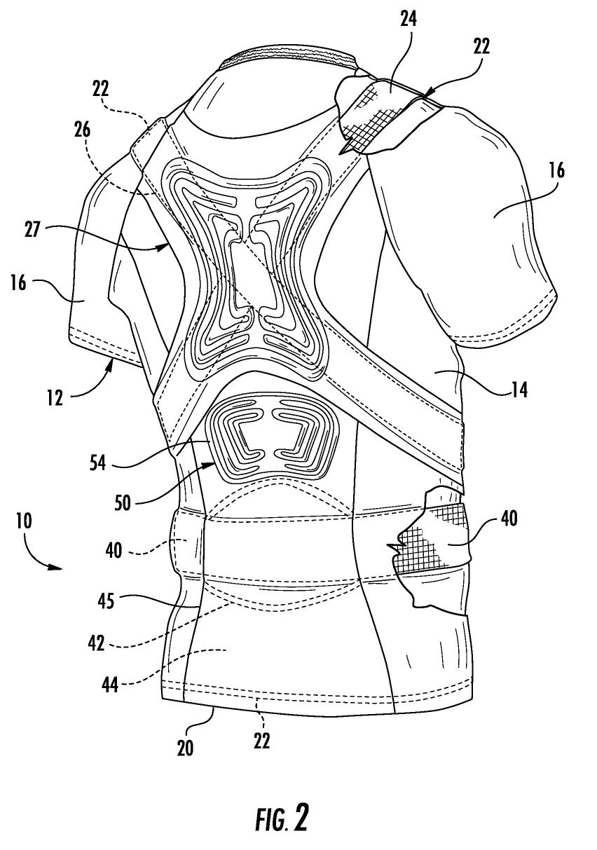

The load distribution garment 10 includes a shirt 12 (FIGS. 1 and 2). The shirt 12 may be made from a suitable “shirting fabric” such as cotton or polyester or blends thereof. In one embodiment, the shirt 12 is made from a moisture wicking fabric for comfort since it is designed to be worn under a ballistic vest. Another suitable material is polyester, with an elastic fit. The shirt 12 has an outside surface that is presented outward, away from the user, when the shirt is being worn by the user.

The particular shirt 12 that is illustrated is in the form of a tee shirt having a torso portion 14 and two sleeves 16. The torso portion 14 is preferably although not necessarily sized, or the overall size of the shirt 12 is selected, so that the torso portion is snug on the wearer's torso. This type of fit can help to prevent the shirt 12 from shifting on the user. In the illustrated embodiment, the torso portion 14 of the shirt 12 has a front zipper 18 to enable donning and doffing of the shirt 12. Alternatively, a pullover shirt can be provided. The lower edge 20 of the torso portion 14 of the shirt 12 is preferably provided with internal soft elastic and a silicone grip strip 21 that helps keep the shirt tall from rising up.

The garment 10 includes two posture control straps, or posture correction straps, designated 22 (FIG. 2). Each posture correction strap 22 has an upper end 24 fixed to a collarbone area of the shirt 12. Each strap 22 extends from its upper end 24 down the hack of the shirt 12 and laterally across the back. The straps 22 extend through an upper strap channel 28 that is formed underneath an extra layer of fabric material 27 located on the outside of the torso portion 14 of the shirt 12. The lower portion of each strap 22 wraps around forward to the opposite side waist area. The lower end of each posture correction strap 22 projects outward from the upper strap channel and terminates in a Velcro hook element 30.

The posture correction straps 22 are elastic. The Velcro hook element 30 on the lower end of each posture correction strap 22 is engageable with a Velcro loop area 32 (FIG. 1) that is provided on the front waist area of the torso portion 14 of the shirt 12. Because all hut the ends of the straps 22 are contained within the upper strap channel 26, the straps 22 are safely covered up and are constrained from moving away from the shirt 12.

When the posture correction straps 22 are pulled tight and secured to the Velcro loop area 32, they act to pull back on the shoulders of the wearer. This helps the wearer to maintain better posture, which makes it more comfortable to wear a heavy outer garment 10 such as a ballistic vest for a longer period of time without tiring. In addition, the tightening of the posture correction straps 22 helps to keep the garment 10 snug on the wearer's torso, so that the load of the ballistic vest 100 is better transferred to the torso in the manner described below.

The garment 10 also includes two waist straps 40. Each waist strap 40 has one end fixed to a padded lumbar back support 42 (FIG. 2). The waist straps 40 extend laterally outward from the lumbar back support 42 and around the torso through a lower strap channel 44 that is formed from an extra layer 45 of fabric material on the outside of the torso portion 14 of the shirt 12. Each waist strap 40 extends out of the lower strap channel 44 and terminates in a Velcro hook element 46. The Velcro hook element 48 is engageable with the Velcro loop area 32 on the front waist area of the shirt 12 (FIG. 1).

The waist straps 40 are elastic. When the waist straps 40 are pulled tight and secured to the Velcro loop area 32, they help the wearer to maintain better posture, which makes it more comfortable to wear a heavy outer garment 10 such as a ballistic vest, for a longer period of time, without tiring. In addition, the tightening of the waist straps 40 helps to keep the garment 10 snug on the wearer's torso, so that the load of the ballistic vest 100 is better transferred to the torso. Because all but the ends of the waist straps 40 are contained within the lower strap channel 44, the straps 40 are safely covered up and are constrained from moving away from the shirt 12.

The load distribution garment 10 includes a plurality of interface grips 50 (FIGS. 1-5). Each interface grip 50 is an exterior part of the garment 10, on the outside surface of the shirt 12. Each grip 50 presents a sticky or tacky surface to any object that contacts the outside of the shirt 12 at that area, in order to increase friction between the shirt and the object.

In the illustrated embodiment, each interface grip 50 is made from a sticky or tacky material that is fixedly positioned on the outside surface of the material of the shirt 12. The material of the interface grips 50 has a significantly higher coefficient of friction than the material of the shirt 12. One suitable material is silicone. The silicone may be disposed in a particular pattern as shown in the drawings, or in another pattern, or may be in one contiguous piece or patch.

One selected area 52 for an interface grip 50, in the illustrated embodiment, is the upper back of the torso portion 14 of the shirt 12 (FIG. 2). Another selected area 54, in the illustrated embodiment, is the lower back of the torso portion 14 of the shirt 12. Another selected area 58, in the illustrated embodiment, is the waist area of the front of the torso portion 14 of the shirt 12 (FIG. 1). In other embodiments, different numbers of interface grips 50 may be provided, and they may be placed at other locations on the garment 10.

When the ballistic vest 100 is worn by a user without a load distribution garment 10, it normally rests on the shoulders and collarbone area of the user. In such a case, the weight of the vest 100 is carried primarily by the shoulder straps 106, and thus is transmitted primarily to the shoulders of the user, not to the core.

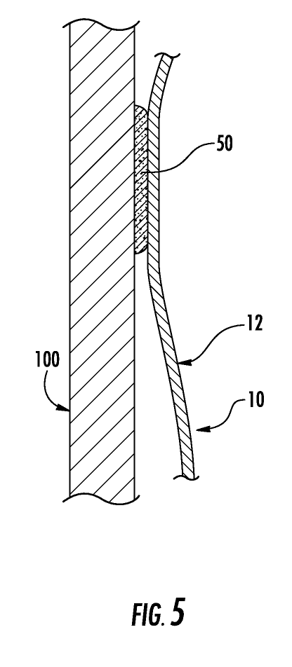

When the ballistic vest 100 is worn over the load distribution garment 10, the vest is preferably snugged up on the user's torso, against the outer surface of the load distribution garment 10, using the standard straps (not shown) that are built into the vest. When the vest 100 is thus tightened on the user's torso, as illustrated schematically in FIG. 5, the inside surface of the vest engages the tacky areas (interface grips 50) on the front and back of the torso portion 14 of the garment 10. This engagement between the vest 100 and the grips 50 increases the friction between the garment 10 and the vest 100, and tends to hold the vest in place at those areas, specifically by resisting downward movement of the vest along the torso of the user. As a result, some of the bad of the vest is transferred to the garment 10, through the grips 50, onto the core of the user. This load distribution reduces the apparent load on the shoulder and collarbone area of the user, making it more comfortable to wear the vest 100 for a longer period of time without tiring.

1. Apparatus comprising
a ballistic vest configured to be worn by a user, the vest having an inside vest surface; and
a load distribution garment configured to be worn by the user under the vest, the load distribution garment having an outside garment surface;
the load distribution garment including a shirt having an outside surface, the outside surface of the shirt forming a first portion of the outside garment surface of the load distribution garment;
the load distribution garment including interface grips located on the outside surface of the shirt, the interface grips having a significantly higher coefficient of friction than the shirt, the interface grips forming a second portion of the outside garment surface of the load distribution garment;
the inside surface of the vest engaging the interface grips on the outside garment surface of the load distribution garment, the engagement of the inside vest surface with the interface grips resisting movement of the vest relative to the load distribution garment.

2. Apparatus as set forth in claim 1 wherein the locations of the interface grips on the outside of the shirt are selected to enable vertically downward load of the vest to be transferred to the shirt at one or more locations other than the user's shoulders and collarbone area.

3. Apparatus as set forth in claim 2 further including tightenable straps on the shirt that help to keep the shirt snug on the user's torso thereby to help ensure that loads placed on the shirt by the vest are transferred to the user's torso.

4. Apparatus as set forth in claim 3 wherein the tightenable straps include posture control straps that extend rearward over the shoulders of the shirt and down the back of the shirt and have lower ends that are securable to the front of the shirt thereby to pull back on the shoulders of the user.

5. Apparatus as set forth in claim 4 wherein the tightenable straps include waist straps that extend laterally from the back of the shirt around the sides of the shirt and have ends that are securable to the front of the shirt to keep the shirt snug on the waist portion of the user's torso thereby to help transfer loads placed on the shirt by the vest, to the user's torso.

6. Apparatus as set forth in claim 3 wherein the straps extend through channels on the shirt.

7. A load distribution garment configured to be worn under a ballistic vest by a user, the vest having an inside vest surface, the load distribution garment comprising:
a shirt made of a shirting material, the shirt having an outside surface;
interface grips located on the outside surface of the shirt, the interface grips having a significantly higher coefficient of friction than the shirt, the interface grips forming a portion of the outside surface of the shirt; and
the inside surface of the vest engaging the interface grips on the outside surface of the shirt, the engagement of the inside vest surface with the interface grips resisting movement of the vest relative to the shirt.

8. Apparatus as set forth in claim 7 wherein the locations of the interface grips are selected to enable vertically downward load of the vest to be transferred to the shirt at one or more locations other than the user's shoulders and collarbone area.

9. Apparatus as set forth in claim 8 further including tightenable straps on the shirt that help to keep the shirt snug on the user's torso thereby to help ensure that loads placed on the shirt by the vest are transferred to the user's torso.

10. Apparatus as set forth in claim 9 wherein the tightenable straps include posture control straps that extend rearward over the shoulders of the shirt and down the back of the shirt and have lower ends that are securable to the front of the shirt thereby to pull back on the shoulders of the user.

11. Apparatus as set forth in claim 10 wherein the tightenable straps include waist straps that extend laterally from the back of the shirt around the sides of the shirt and have ends that are securable to the front of the shirt to keep the shirt snug on the waist portion of the user's torso thereby to help transfer loads placed on the shirt by the vest, to the user's torso.

12. Apparatus as set forth in claim 9 wherein the straps extend through channels on the shirt.
